(19) United States
(12) Patent Application Publication
(10) Pub. No.: US 2017/0258962 A1
(43) Pub. Date: Sep. 14, 2017

(54) BONE OR OSTEOCHONDRAL TISSUES AND USES THEREOF

(71) Applicants: Colorado State University Research Foundation, Fort Collins, CO (US); The Regents of the University of California, LaJolla, CA (US)

(72) Inventors: Suzanne M. Tabbaa, Fort Collins, CO (US); David D. Frisbie, Fort Collins, CO (US); C. Wayne McIlwraith, Loveland, CO (US); Robert L. Sah, San Diego, CA (US); William D. Bugbee, San Diego, CA (US)

(73) Assignees: Colorado State University Research Foundation, Fort Collins, CO (US); The Regents of the University of California, LaJolla, CA (US)

(21) Appl. No.: 15/453,640

(22) Filed: Mar. 8, 2017

Related U.S. Application Data
(60) Provisional application No. 62/305,290, filed on Mar. 8, 2016.

(51) Int. Cl. A61L 27/36; A61L 27/56; A61L 27/38

(52) U.S. Cl. CPC A61L 27/3687; A61L 27/3821; A61L 27/3847; A61L 27/56; A61L 2430/02; A61L 2400/12

(57) ABSTRACT

The present disclosure provides modified bone and osteochondral tissues, methods of preparing modified bone and osteochondral tissues, and methods of using the modified bone or osteochondral tissues to repair bone or osteochondral defects in a subject.

### CROSS REFERENCE TO RELATED APPLICATIONS

This application claims the benefit of U.S. Provisional Application No. 62/305,290, filed Mar. 8, 2016, the disclosure of which is hereby incorporated by reference in its entirety.

### FIELD OF THE INVENTION

The disclosure relates to modified bone or osteochondral tissue, and methods of use and preparation thereof.

### BACKGROUND OF THE INVENTION

Bone and cartilage injuries are highly prevalent and persistent problems in orthopedics. When left untreated, these injuries may contribute to joint irritation and inflammation over time. To repair bone and cartilage injuries, a surgeon may elect to harvest bone or cartilage tissue from the affected patient or from another donor source. The harvested tissue is processed into a graft that can be implanted into the damaged area.

In recent years, improvements to the procurement and storage processes of grafts have led to their increased use to treat bone and cartilage injuries. Although the clinical success rate for such procedures is generally high, later complications that may occur are associated with inadequate bone integration and formation at the graft site. Also, as demand for graft implantation to treat bone and cartilage injuries increases, the availability of donor grafts is limited due to suboptimal storage conditions. Current procedures for processing donor grafts are limited by the need to preserve functional properties of the graft. Hence, there is a need in the art for better graft processing techniques and graft compositions.

### SUMMARY OF THE INVENTION

In one aspect, the present disclosure provides a modified ex vivo bone or osteochondral tissue, wherein at least 15%, 20%, 25%, etc., of the residual lipids in the bone or osteochondral tissue have been depleted compared to a non-modified bone or osteochondral tissue.

In another aspect, the present disclosure provides a modified ex vivo bone or osteochondral tissue comprising bone nano-channels, micro-channels, or a combination of nano-channels and micro-channels.

In one embodiment, the modified ex vivo bone or osteochondral tissue may also be combined with or incubated with one or more osteogenic agents.

In another aspect, the present disclosure provides methods of modifying a bone or osteochondral tissue. The methods comprise incubating the bone or osteochondral tissue with one or more lipases under conditions suitable for activity of the one or more lipases, wherein after incubation, at least 15%, 20%, 25%, etc., of the residual lipids in the modified bone or osteochondral tissue are depleted compared to a non-modified bone or osteochondral tissue.

In yet another aspect, the present disclosure provides methods of repairing a bone or osteochondral defect in a subject. The method comprises implanting a modified bone at the site of a bone defect in the subject, or implanting a modified osteochondral tissue at the site of an osteochondral defect in the subject. In certain embodiments, at least 15%, 20%, 25%, etc., of the residual lipids in the modified bone or modified osteochondral tissue have been depleted compared to a non-modified bone or osteochondral tissue. In other embodiments, the modified bone or modified osteochondral tissue comprise nano-channels, micro-channels, or a combination of nano-channels and micro-channels. In other embodiments, the modified bone or modified osteochondral tissue may be combined with or incubated with one or more osteogenic agents.

Other aspects and iterations will be apparent herein.

### BRIEF DESCRIPTION OF THE FIGURES

The application file contains at least one drawing executed in color. Copies of this patent application publication with color drawing(s) will be provided by the Office upon request and payment of the necessary fee

FIG. 1 depicts methods of preparing bone or osteochondral tissue depleted of residual lipids with lipase. Osteochondral cores (diameter=8-10 mm, bone thickness=8-10 mm) were harvested from the medial trochlear ridge of joints from recently-deceased adult horses. Fresh osteochondral cores were incubated in an aqueous solution that contained physiological buffers with the lipase enzymes (incubation at 10 mg/mL at 37° C. for 4 hour), with moderate agitation. Following lipase incubation, the osteochondral cores were soaked and washed by traditional lavage with saline at a flow rate of 1.5 L/min. Lipase solution was collected to measure the release of triglycerides using a triglyceride assay. Cartilage was removed from the lipase-treated osteochondral core to measure chondrocyte viability using a LIVE/DEAD assay. The subchondral bone from the lipase-treated osteochondral core is used to analyze adipose marrow removal by a variety of qualitative and quantitative methods.

FIGS. 2A, B, and C depict images demonstrating that lipase treatment of osteochondral cores is effective in removing adipose marrow from subchondral bone. The extent of remaining residual marrow elements in the subchondral bone was measured using contrast-enhance micro-computed tomography (μCT) and shown here in a representative image of a transverse slice. Marrow removal using μCT with and without contrast agent is indicated by less marrow (black) and greater infiltration of contrast-agent (gray). (FIG. 2A) μCT images of control samples show minimal infiltration of contrast agent (gray). (FIG. 2B) μCT images of lavage-treated samples show minimal infiltration of contrast agent (gray). (FIG. 2C) μCT images of lipase-treated samples show extensive infiltration of contrast agent (gray).

FIGS. 3A and 3B shows data demonstrating that lipase treatment of osteochondral cores increases removal of lipid from the tissue. The extent of remaining lipid in control (CTRL), lavage-treated (LAV), and lipase-treated (LIP) samples were quantified and compared (* indicates p<0.05). (FIG. 3A) To quantify lipid removal in the three treatment groups, an Oil Red O extraction and quantification procedure, (stain specific for neutral lipids) was used to quantify the amount of residual neutral lipid. Lipase-treated osteochondral cores show significantly less neutral lipid content compared to the control and lavage-treated groups. (FIG. 3B) To further quantify lipid removal, adipose marrow breakdown in osteochondral cores during lipase treatment was quantified by measuring the amount of released triglycerides in the lipase solution after the incubation period using a commercially available colorimetric assay (Infinity Triglycerides (Sigma)). Lipase-treated osteochondral cores show significantly more triglyceride release into the cleansing solution compared to the control, untreated group.

FIGS. 4A, 4B, and 4C illustrates multidirectional fabrication of bone nano- and micro-channels. Shown is an example of the direction, size, and depth of channels that could be cut into a bone or osteochondral tissue. (FIG. 4A) A 3D view of a harvested core tissue containing nano- and micro-channels. (FIG. 4B) A side view of a harvested core tissue containing nano- and micro-channels. (FIG. 4C) A transverse view of a harvested core tissue containing nano- and micro-channels.

FIG. 5 depicts an illustration of methods of modifying the bone architecture of osteochondral grafts with bone channels (e.g., nano- and/or micro-bone channels) and removing residual lipids from the modified osteochondral graft. Osteochondral grafts (e.g., diameter=8-10 mm, bone thickness=8-10 mm) may be harvested from medial trochlear ridge of joints from recently-deceased adult horses. Following harvest, channels in the transverse, radial, perpendicular, as well as defined orientation through trabeculae of various diameters (50 nm-100 μm) may be generated, e.g., by laser technology. As illustrated, the modified osteochondral allograft may then be incubated in an aqueous solution that contains physiological buffers with lipase enzymes (e.g., 10 mg/mL). The modified osteochondral cores may be incubated for 4 hour at 37° C. in the lipase solution with moderate agitation. Following lipase incubation, the osteochondral cores may be soaked and washed by traditional lavage with saline, e.g., at a flow rate of 1.5 L/min. Adipose marrow-depleted cores may then be incubated with autologous cells previously isolated from the patient for an adequate time prior to implantation.

### DETAILED DESCRIPTION OF THE INVENTION

In certain aspects, the present invention encompasses modified ex-vivo bone or osteochondral tissues. As used herein, “bone or osteochondral tissues” refers to a three-dimensional portion, plug, or core of bone (e.g. bone graft), or a tissue comprising a combination of bone and cartilage (e.g. osteochondral tissue from femoral condyles, trochlea, patella, femoral heads, etc.), that is harvested from a donor. In certain aspects, such a bone or osteochondral tissues is modified, ex vivo, by a method of this disclosure. In the context of the present disclosure, osteochondral tissue includes whole osteochondral and bone tissue blocks, and well as processed portions of whole tissue blocks including osteochondral allografts (OCA), plugs, cores and grafts. The concepts and methods described herein apply equally to whole tissue blocks, allografts, plugs cores and grafts.

In certain aspects, the disclosure provides modified ex-vivo bone or osteochondral tissues, wherein the bone or tissues have been depleted of residual lipids. In certain aspects, the residual lipids have been depleted by incubation with one or more lipase, as described herein. In certain embodiments, the modified ex-vivo bone or osteochondral tissues having depleted residual lipids may therefore comprise one or more lipase distribution structural features in the bone or osteochondral tissue. In certain embodiments, the lipase distribution structural features may be holes, perforations, channels (including nano-channels, micro-channels, or a combination of nano-channels and micro-channels as described herein), or combinations thereof.

In other aspects, the disclosure provides modified ex-vivo bone or osteochondral tissues, wherein the bone or tissues comprise bone nano-channels, micro-channels, or a combination of nano-channels and micro-channels in the bone or osteochondral tissue.

In certain embodiments, the modified bone or osteochondral tissue of the present disclosure may be combined with or incubated with one or more osteogenic agents.

In certain aspects, the disclosure provides methods for modifying a bone or osteochondral tissue. In certain embodiments, the modification depletes residual lipids in the bone or osteochondral tissue. In other embodiments, the modification may comprise inclusion of bone nano-channels, micro-channels, or a combination of nano-channels and micro-channels in the bone or osteochondral tissue. In yet other embodiments, the modification may comprise a combination of a depletion of residual lipids and an inclusion of bone channels in the bone or osteochondral tissue. In certain embodiments, the bone channels may be bone nano-channels, micro-channels, or a combination of nano-channels and micro-channels.

Modified bone or osteochondral tissue of the present disclosure may be utilized for surgical implantation in a subject. For instance, a modified bone or osteochondral tissue of the present disclosure may be used to repair a bone or osteochondral defect in a subject. Furthermore, the present invention provides methods for increasing the length of time a modified bone or osteochondral tissue may be stored ex vivo compared to a non-modified bone or osteochondral tissue, methods for improving chondrocyte viability, and for improving the osteoconductive property of a bone by removing or decreasing inhibitors to bone formation.

### I. Modified Ex Vivo Bone or Osteochondral Tissue

One aspect of the invention provides a modified bone or osteochondral tissue, wherein the bone or osteochondral tissue is depleted of residual lipids. As used herein, “residual” refers to lipids that remain in a bone or osteochondral tissue after harvest from a donor. As used herein, “depletion” means that at least about 15%, at least about 20%, at least about 25%, at least about 35%, at least about 45%, at least about 55%, at least about 65%, at least about 75%, at least about 85%, at least about 95%, of the residual lipids in the bone or osteochondral tissue are removed compared to a control bone or osteochondral tissue that is not treated by a method described herein. Control bone or osteochondral tissue (e.g. non-modified bone or osteochondral tissue) should be the same size, shape, and type of bone or tissue as the modified bone or tissue.

In some embodiments, a modified bone or osteochondral tissue is prepared using a method described herein such that about 25% to about 100%, about 35% to about 100%, about 45% to about 100%, about 55% to about 100%, about 65% to about 100%, about 75% to about 100%, about 85% to about 100%, or about 95% to 100% of the residual lipids in the modified bone or osteochondral tissue are removed compared to a non-modified bone or osteochondral tissue.

In one embodiment, the residual lipids depleted from a modified bone or osteochondral tissue using a method of the present disclosure may comprise long chain fatty acids. For instance, non-limiting examples of long chain fatty acids that may be depleted include palmitic acid, stearic acid, oleic acid and linoleic acid.

In still another embodiment, a modified bone or osteochondral tissue may be depleted of residual lipids comprised of residual adipose marrow. As used herein, “adipose marrow” refers to the lipid component of bone marrow. As used herein, “residual” refers to the amount of adipose marrow that remains in a bone or osteochondral tissue after harvest from a donor. In one embodiment, a modified bone or osteochondral graft is depleted of at least about 15%, at least about 20%, at least about 25%, at least about 35%, at least about 45%, at least about 55%, at least about 65%, at least about 75%, at least about 85%, or at least about 95%, of the residual bone marrow compared to a non-modified bone or osteochondral tissue. In some embodiments, a modified bone or osteochondral tissue is prepared using a method described herein such that about 25% to about 100%, about 35% to about 100%, about 45% to about 100%, about 55% to about 100%, about 65% to about 100%, about 75% to about 100%, about 85% to about 100%, or about 95% to about 100% of the residual bone marrow is depleted compared to a non-modified bone or osteochondral tissue.

In certain embodiments, a modified bone or osteochondral tissue of the disclosure may be an ex vivo modified bone or osteochondral tissue, wherein the bone or osteochondral tissue is depleted of residual adipose marrow.

In other embodiments, a modified bone or osteochondral tissue of the present disclosure may comprise bone nano-channels, micro-channels, or a combination of nano-channels and micro-channels. The nano and/or micro channels provide for chemical and biological wicking, penetration and/or infiltration into the bone or osteochondral tissue. As used herein, the term “nano-channel” refers to channels at least about 50 nm, at least about 60 nm, at least about 70 nm, at least about 80 nm, or at least about 90 nm in diameter in a modified bone or osteochondral tissue. As used herein, the term “micro-channel” refers to channels between about 1 μm and about 100 μm, between about 10 μm and about 100 μm, between about 20 μm and about 100 μm, between about 30 μm and about 100 μm, between about 40 μm and about 100 μm, etc. in diameter in a modified bone or osteochondral graft.

In certain embodiments, a modified bone or osteochondral tissue may comprise micro-channels oriented in an array of longitudinal and radial channels of less than 100 μm, e.g., ranging in size from about 20 μm to about 100 μm, 30 μm to about 90 μm, about 40 to about 80 μm, etc. In certain embodiments, the array will include at least 4 channels of about 40 μm in diameter, at least 4 channels of about 60 μm in diameter, and at least 4 channels of about 80 μm in diameter.

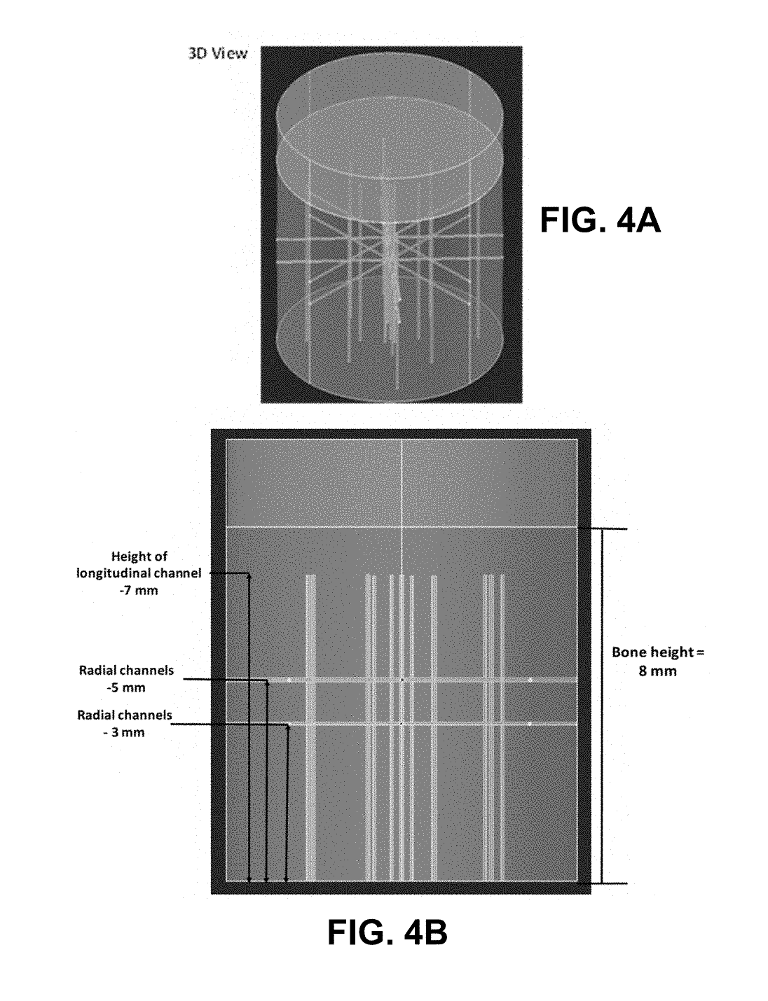

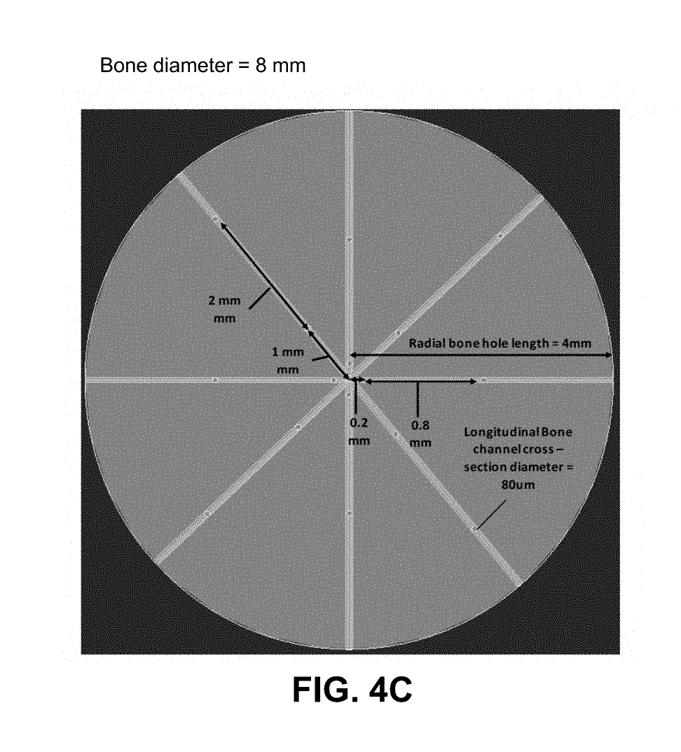

The bone nano-channels or micro-channels may extend the length of modified bone or osteochondral tissue, or may extend for only a portion of the modified bone or osteochondral tissue. For instance, with reference to FIGS. 4A-4B, the nano and micro channels may extend across the full distance side to side, approximately three-quarters distance top to bottom, and may generally intersect at a mid-point in the center on the cross-plane.

In certain embodiments, a modified bone or osteochondral tissue of the present disclosure may comprise longitudinal, radial, transverse and/or perpendicular bone nano-channels, micro-channels, or a combination of nano-channels and micro-channels. In one embodiment, the bone nano-channels, micro-channels, or a combination of nano-channels and micro-channels may be contained in the underlying bone without extending into cartilage. In another embodiment, the bone nano-channels, micro-channels, or a combination of nano-channels and micro-channels may occupy one or more portions of the deep to intermediate zone of the cartilage and may not occupy the superficial zone of the cartilage. In yet another embodiment, the modified bone or osteochondral tissue may contain bone nano-channels, micro-channels, or a combination of nano-channels and micro-channels along the whole length of the bone portion up to the cartilage and bone interface. In still another embodiment, the bone nano-channels, micro-channels, or a combination of nano-channels and micro-channels may be directly contacted with the bone portion of a bone or osteochondral tissue, and may end at the deep, intermediate, or superficial zone of the cartilage.

In certain embodiments, a modified bone or osteochondral graft may comprise micro-channels oriented in an array of longitudinal and radial channels of between about 1 μm and about 100 μm, e.g., ranging in size from about 40 μm to about 80 μm. In certain embodiments, the array will include at least 4 channels of 40 μm, at least 4 channels of 60 μm, and at least 4 channels of 80 μm.

In some embodiments, a modified bone or osteochondral tissue of the disclosure may comprise a combination of, e.g., longitudinal and radial bone channels, including nano and/or micro bone channels, in an array to provide a defined bone channel architecture designed to facilitate chemical and bio-infiltration into the bone or osteochondral tissue. In general, the bone channels, e.g., micro bone channels, may form a bone channel architecture that provides sufficient wicking access to the interior of the bone or osteochondral tissue such that chemical and/or biological materials are able to penetrate or infiltrate substantially all of the bone or osteochondral tissue under conditions of use. Without intending to be limited by theory, improved chemical and bio-infiltration into the bone or osteochondral tissue may provide for improved nutrient and cellular infiltration and repopulation of osteogenic cells in vivo upon surgical implantation in accordance with embodiments of present disclosure.

In some embodiments, lipase distribution structural features may be used in combination with modifications described herein related to the depletion of residual lipids. In these embodiments, the lipase distribution structural features may be created in the bone or osteochondral tissue either before or after depletion of residual lipids. As used herein, the term “lipase distribution structural feature” refers to an opening made in the bone or osteochondral tissue. Such an opening may include, but are not limited to, holes, perforations, channels (including micro- and nano-channels as described herein) and combinations thereof. In embodiments wherein the lipase distribution structural features are formed prior to depletion of residual lipids, the lipase distribution structural features may enhance penetration of lipases (as described herein) into the bone or osteochondral tissue so as to facilitate removal of residual lipids. In certain embodiments, when used in combination with methods to deplete residual lipids, the lipase distribution structural features may be formed with any suitable size, shape and configuration to enhance penetration of lipases into the bone or osteochondral tissue and/or facilitate removal of residual lipids. For example, the lipase distribution structural features may be formed using standard drilling, punching, or lasering techniques known in the art, and may have sizes ranging from about 1 μm to about 1 mm in diameter, but are not so limited. Further, the lipase distribution structural features may have an array of longitudinal and radial openings, as described herein in connection with bone channels.

More particularly, in some embodiments, a modified bone or osteochondral tissue of the disclosure may comprise a combination of different types of lipase distribution structural features, e.g., longitudinal and radial drilled holes in combination with punched perforations at the surface and laser drilled bone channels into the interior, including nano and/or micro bone channels, in an array to provide a defined structural architecture designed to facilitate chemical and bio-infiltration into the bone or osteochondral tissue. In general, the lipase distribution structural features may form a lipase distribution architecture that provides sufficient wicking access to the interior of the bone or osteochondral tissue such that chemical and/or biological materials including the one or more lipases are able to penetrate or infiltrate substantially all of the bone or osteochondral tissue under conditions of use. Without intending to be limited by theory, improved chemical and bio-infiltration into the bone or osteochondral tissue may provide for improved cleaning and lipase reduction, in accordance with embodiments of present disclosure.

Methods of harvesting a bone or osteochondral tissue from a donor are known in the art. As used herein, “donor” refers to an animal that has a skeleton. In certain embodiments, the donor may be a mammal, and in some embodiments, the donor may be a human. In certain embodiments, a donor may be a livestock animal (horse, cow, pig, sheep, etc.) or a companion animal (dog, cat, etc.). In a particular embodiment, the donor may be an equine. In each of the above embodiments, the donor may be a cadaver. The harvested bone or osteochondral tissue may include whole or partial donor bone or osteochondral tissue, such as but not limited to whole or partial femoral condyles, hemi-condyles, femoral trochlea, humeral heads, distal tibia, femoral head, talus, patella, etc. Any suitable size and shape of donor material may be obtained, depending on the intended use and further processing needs, as recognized by those of skill in the art. By way of non-limiting example, an average size for donor osteochondral tissue may be greater than 20 cm², with an average area of an adult human patella being ˜12 cm².

In each of the above embodiments, a bone or osteochondral tissue may be harvested from a donor that will subsequently receive the modified bone or osteochondral tissue (e.g. an autograft). Alternatively, in each of the above embodiments, a bone or osteochondral tissue may be harvested from a donor other than the recipient (e.g. an allograft). For instance, in each of the above embodiments, a bone or osteochondral tissue may be harvested from a cadaveric donor. A bone or osteochondral tissue harvested from a cadaveric donor is typically stored ex vivo until needed to repair a defect in a subject. Such bone or osteochondral tissues may be modified before or after storage. Methods of storing bone or osteochondral tissues ex vivo are known in the art. By way of non-limiting example, bone or osteochondral tissues may be stored at about 24° C. or 37° C., or at lower temperatures, e.g., 4° C., −40° C. or lower if desired.

In some embodiments, the modified bone or osteochondral tissue may be combined with or incubated with one or more osteogenic agents.

For instance, a modified bone or osteochondral tissue may be incubated with osteogenic cells in vitro or ex vivo using techniques commonly known in the art. The cells may be differentiated or undifferentiated. Suitable, non-limiting examples of cells may include chondrocytes, osteoblasts, bone marrow cells, mesenchymal stem cells, adipose derived stem cells, amniotic stem cells, progenitor cells, etc. In certain embodiments, a modified bone or osteochondral tissue may be combined or incubated with cells isolated from the intended recipient of the graft. In other embodiments, a modified bone or osteochondral tissue may be incubated with cells from sources other than the intended recipient of the bone or osteochondral tissue. In some embodiments, a modified bone or osteochondral tissue may be combined or incubated with bone marrow cells, mesenchymal stem cells, or a combination thereof. For instance, a modified bone or osteochondral tissue may be incubated with mesenchymal stem cells from various sources including bone marrow aspirate, adipose tissue, and peripheral blood.

In certain embodiments, a modified bone or osteochondral tissue may be further combined with osteogenic agents meant to improve bone or osteochondral tissue function in the recipient. Suitable elements are known in the art and may include allogenic cells, osteoconductive factors, and osteoinductive factors. For instance, in some embodiments, a modified bone or osteochondral tissue may be combined with one or more growth factors. Non-limiting growth factors may include, but are not limited to, transforming growth factor-beta (TGFβ), fibroblast growth factor (FGF) (e.g., FGF2, FGF5), bone morphogenetic protein (BMP) (e.g., BMP2, BMP4, BMP6, BMP7), platelet derived growth factor (PDGF), and insulin-related growth factor (IGF) (e.g., IGF1, IGF2).

### II. Methods

Methods of the present disclosure include methods of modifying a bone or osteochondral tissue such that residual lipids are depleted, methods of forming lipase distribution structural features, methods of forming bone channels in a bone or osteochondral tissue, methods of extending storage of a bone or osteochondral tissue, methods of improving chondrocyte viability in a bone or osteochondral tissue, methods of improving the osteoconductive property of bone by removing inhibitors to bone formation. Methods for combining modified bone or osteochondral tissue with an osteogenic agents, and related methods of use are also provided. Additionally, methods of repairing a bone or osteochondral defect by implanting, in a subject, a modified bone or osteochondral graft are described herein.

### (a) Preparing a Lipid Depleted Modified Bone or Osteochondral Graft

One embodiment of the present disclosure provides methods of modifying a bone or osteochondral tissue to deplete residual lipids. The method comprises incubating a bone or osteochondral graft with one or more lipases under conditions suitable for activity of the one or more lipases, wherein after incubation, at least about 15%, at least about 20%, at least about 25%, etc., of the residual lipids in the modified bone or osteochondral tissue are depleted compared to a non-modified bone or osteochondral tissue.

In another embodiment, the modified bone or osteochondral tissue may be combined or incubated with an osteogenic agent, such as a cell as described herein. In certain embodiments, the osteogenic agent may be combined into the modified bone or osteochondral tissue to fill the depleted bone marrow voids in the bone.

(i) Incubation with One or More Lipases

A method of the present disclosure comprises incubating a bone or osteochondral tissue with one or more lipases under conditions suitable for activity of the one or more lipases, wherein after incubation, at least 15%, at least about 20%, at least about 25%, etc., of the lipids in the modified bone or osteochondral tissue are depleted compared to a non-modified bone or osteochondral tissue.

As used herein, a “lipase” refers to an enzyme that catalyzes the hydrolysis of lipids. In connection with the present methods, any suitable mammalian or non-mammalian lipase active to degrade the lipid component of bone marrow may be used. For instance, a suitable lipase may hydrolyze residual triglycerides in a harvested osteochondral tissue. In some embodiments, a lipase may be a triacylglycerol lipase with an EC number of 3.1.1.3. By way of non-limiting example, suitable lipases may be selected from mammalian pancreatic lipase, a Candida rugosa lipase, a Geotrichum candidum lipase, and a Chromobacterium viscosum lipase, and combinations thereof. One of skill in the art will understand that the amount of the one or more lipases used may be determined by the volume of bone or osteochondral tissue to be modified. In certain embodiments, a bone or osteochondral tissue may be incubated with at least two, at least three, at least four, or at least five lipases.

Generally speaking, a bone or osteochondral tissue may be incubated with one or more lipases in an aqueous media under conditions suitable for activity of one or more lipases. In one embodiment, a bone or osteochondral tissue may be incubated in an aqueous media where the pH may be between about 5 to about 10, about 5.5 to about 9.5, about 6 to about 9, or about 6 to about 8.5. In preferred embodiments, the pH may be about 7.2 to about 8.0, or about 7.4 to about 7.6. In some embodiments, the osmotic pressure of the aqueous media may be between about 50-500 mosm/kg or between about 100-350 mosm/kg. In other embodiments, the osmotic pressure of the aqueous medium may be between about 250-350 mosm/kg, or between about 280-300 mosm/kg. In certain embodiments, the salt concentration of the aqueous media may be about 100 to about 300 mM NaCl. In some embodiments, the salt concentration of the aqueous media may be about 150 mM NaCl. Other suitable salts may be used, including those typically used for cell and tissue preservation medium, e.g., calcium chloride, potassium chloride, sodium bicarbonate, magnesium chloride, etc. In certain embodiments, the aqueous medium may comprise tissue culture media, blood, or bone or osteochondral fluid.

Generally speaking, incubation conditions should be suitable for activity of the one or more lipases. In one embodiment, a bone or osteochondral tissue may be incubated with one or more lipases under temperatures suitable for activity of the one or more lipases. In another embodiment, a bone or osteochondral tissue may be incubated with one or more lipases under temperatures within a physiological range. In another embodiment, a bone or osteochondral tissue may be incubated with one or more lipases under temperatures from about −80° C. to about 80° C., from about −80° C. to about 45° C., from about 4° C. to about 80° C., from about 4° C. to about 45° C., from about 30° C. to about 39° C., etc. In other embodiments, the graft may be incubated with one or more lipases for a period of time preferably of about 1 minute to about 72 hours, from about 15 minutes to about 24 hours, from about 30 minutes to about 24 hours, from about 1 hours to about 24 hours, from about 1 hours to about 12 hours, from about 1 hours to about 8 hours, from about 1 hours to about 4 hours, and from about 1 hours to about 2 hours. After a suitable incubation, at least about 15%, at least about 20%, at least about 25%, and up to 100%, of the residual lipids in the modified bone or osteochondral tissue are depleted compared to a non-modified bone or osteochondral tissue.

In certain embodiments, various mechanical, electrical, or magnetic techniques may be used to improve disbursement of the one or more lipases throughout the bone or osteochondral tissue. For example, the bone or osteochondral tissue may be incubated with moderate agitation. Any suitable type and rate of agitation may be used, e.g., about 10 rpm to about 1000 rpm, about 100 rpm to about 500 rpm, etc. In another embodiment, a peristaltic pump, vacuum, sonication, or combination thereof may also be used to improve disbursement of the one or more lipases throughout the bone or osteochondral tissue.

In other embodiments, as described above, the bone or osteochondral tissue may include lipase distribution structural features formed prior to depletion of residual lipids. The lipase distribution structural features may enhance penetration of the one or more lipases into the bone or osteochondral tissue so as to facilitate removal of residual lipids. As described herein, the lipase distribution structural features may be formed with any suitable size, shape and configuration to enhance penetration of lipases into the bone or osteochondral tissue and/or facilitate removal of residual lipids. For example, the lipase distribution structural features may have, by way of non-limiting example, diameters ranging from about 1 μm to about 1 mm. Further, the lipase distribution structural features may have an array of longitudinal and radial openings, as described herein with reference to bone channels. In addition, the lipase distribution structural features may be used in combination with the various mechanical, electrical, or magnetic techniques described herein.

After incubation with one or more lipases, a method of the present disclosure may additionally comprise inhibiting further lipase activity. In one embodiment, lipase activity is inhibited by the addition of a lipase inhibitor to the aqueous media. Methods of inhibiting lipase activity are known in the art and non-limiting examples of lipase inhibitors may include serine protease inhibitors, EDTA, and known inhibitor ions specific to lipase type. In certain embodiments, lipase activity may be inhibited by heat inactivation of the lipase enzyme.

A method of the present disclosure may further comprise washing a bone or osteochondral tissue after incubation with one or more lipases. Non-limiting examples of washing techniques may include pulse lavage, use of a vacuum, or use of a peristaltic pump. In other embodiments, methods of the disclosure may further comprise washing a bone or osteochondral tissue prior to incubation with one or more lipases. Again, non-limiting examples of washing techniques may include pulse lavage, use of a vacuum, or use of a peristaltic pump.

Following incubation of a bone or osteochondral tissue with one or more lipases, a bone or osteochondral tissue may contain at least about 15%, at least about 20%, at least about 25%, at least about 35%, at least about 45%, at least about 55%, at least about 65%, at least about 75%, at least about 85%, or at least about 95%, less residual lipids than a non-modified control. In particular embodiments, a modified bone or osteochondral tissue may be depleted such that about 25% to about 100%, about 35% to about 100%, about 45% to about 100%, about 55% to about 100%, about 65% to about 100%, about 75% to about 100%, about 85% to about 100%, or about 95% to about 100% of the residual adipose marrow is depleted. The amount of residual lipids removed from a modified bone or osteochondral tissue may be measured using assays known to one skilled in the art. Non-limiting examples may include oil red O staining and colorimetric analysis of triglyceride breakdown products.

### (b) Preparing a Channel Modified Bone or Osteochondral Graft

One embodiment of the present disclosure provides methods of modifying a bone or osteochondral tissue to provide bone channels, including nano-channels, micro-channels, or a combination of nano-channels and micro-channels. In another embodiment, the present disclosure provides methods of modifying a bone or osteochondral tissue to provide lipase distribution structural features. The methods described herein to provide bone channels may also be used to provide lipase distribution structural features.

### (i) Fabrication of Nano- and Micro-Sized Channels in a Bone or Osteochondral Tissue.

In some embodiments, a bone or osteochondral tissue of the present disclosure may comprise bone channels, including nano-channels, micro-channels, or a combination of nano-channels and micro-channels. In certain aspects, the bone channels may be longitudinal, radial, transverse and/or perpendicular. In certain embodiments, the bone channels may comprise nano-channels, micro-channels, or a combination of nano-channels and micro-channels.

In one embodiment, the bone channels may be contained in the underlying bone without extending into cartilage. In another embodiment, the bone channels may occupy one or more portions of the deep to intermediate zone of the cartilage and may not occupy the superficial zone of the cartilage. In yet another embodiment, the bone may contain bone channels along the whole length of the bone portion up to the cartilage and bone interface. In still another embodiment, the bone channels may be directly contacted with the bone portion of a bone or osteochondral tissue, and may end at the deep, intermediate, or superficial zone of the cartilage.

More particularly, in some embodiments, a modified bone or osteochondral tissue of the disclosure may comprise a combination of, e.g., longitudinal and radial bone channels in an array to provide a defined bone channel architecture designed to facilitate chemical and bio-infiltration into the bone or osteochondral tissue. In certain embodiments, the bone channels may comprise micro-channels, nano-channels or a combination thereof. Without intending to be limited by theory, improved chemical and bio-infiltration into the bone or osteochondral tissue will provide for improved cleaning and lipase reduction, as well as improved nutrient and cellular infiltration and repopulation of osteogenic cells in vivo upon surgical implantation in accordance with embodiments of present disclosure.

In some embodiments, 1, 2, 3, 4, 5, 6, 7, 8, 9, 10, 15, 20, 25, 30, 35, 40, 45, 50, 60, 70, 80, 90, 100, 150, 200 or more channels may be cut into a bone or osteochondral tissue. Nano-channels, micro-channels, or a combination of nano-channels and micro-channels may be at least about 50 nm, at least about 60 nm, at least about 70 nm, at least about 80 nm, at least about 90 nm at least about 1 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm in diameter in a bone or osteochondral tissue. The number, size and placement of bone channels will depend on the size of the bone or osteochondral tissue, and the desired penetration into the bone or tissue.

In certain embodiments, a modified bone or osteochondral tissue may comprise micro-channels oriented in an array of longitudinal and radial channels of less than 100 μm, e.g., ranging in size from about 20 μm to about 100 μm, 30 μm to about 90 μm, about 40 to about 80 μm, etc. In certain embodiments, the array will include at least 4 channels of 40 μm, at least 4 channels of 60 μm, and at least 4 channels of 80 μm.

In some embodiments, such bone channels may be used in combination with modifications described herein related to depletion of residual lipids. In these embodiments, the channels may be created in the bone or osteochondral tissue either before or after depletion of residual lipids.

Any suitable method for forming the nano-channels, micro-channels, or a combination of nano-channels and micro-channels into the bone or osteochondral tissue may be used. In some embodiments, nano-channels, micro-channels, or a combination of nano-channels and micro-channels may be cut into the bone or osteochondral tissue using a laser cutting apparatus. In certain aspects, laser technology may be used to obtain non-cylindrical, small diameter bone channels. For example, tapered bone channels with a larger inlet and smaller outlet with the desired diameter (e.g.,<100 um) may be prepared using laser technology. For example, in some embodiments, the laser cutting apparatus may be a laser engraver. Non-limiting examples of suitable engraving lasers may include CO₂ engraving lasers, such as the Epilog Zing 30 Watt CO₂ engraving laser.

### (c) Harvesting Tissue for a Bone or Osteochondral Tissue

Generally speaking, methods of harvesting a bone or osteochondral graft suitable for the present disclosure are known in the art. In some embodiments of the present disclosure, the graft donor may be the same as the recipient (e.g. an autograft). In other embodiments, the graft donor may be different from the recipient, but still a member of the same species as the recipient (e.g. an allograft). In still other embodiments, the graft donor may be from a different species as the recipient. (e.g. a xenograft). In certain embodiments, the donor or recipient may be a mammal, and in some embodiments, the donor or recipient may be a human. In certain embodiments, a donor or recipient may be a livestock animal (horse, cow, pig, sheep, etc.) or a companion animal (dog, cat, etc.). In a particular embodiment, the donor may be an equine. In each of the above embodiments, the donor may be a cadaver. Suitable xenograft donors when the recipient is human may include canine, caprine, swine, ovine, bovine and equine donors. Similarly, suitable xenograft donors when the recipient is a livestock or companion animal may include non-similar canine, caprine, swine, ovine, bovine and equine donors.

In some embodiments, a single bone or osteochondral tissue may be harvested from a donor. In other embodiments, multiple bones or osteochondral tissues may be harvested from a single donor. In certain embodiments, 2, 3, 4, 5, 6, or more than 6 bones or osteochondral tissues may be harvested from a single donor.

In each of the above embodiments, bone or osteochondral tissue may be harvested from any appropriate structure that includes cartilage, bone, or a combination of cartilage and bone. The harvested cartilage within an osteochondral tissue may comprise deep, intermediate, or superficial zones of cartilage. The harvested bone within a osteochondral tissue may comprise subchondral bone, cancellous bone, or a combination thereof. In each of the above embodiments, a bone or osteochondral tissue may be harvested from a non-weight bearing surface. For instance, a bone or osteochondral tissue may be harvested from a non-weight bearing portion of a joint. In certain embodiments, a bone or osteochondral tissue may be harvested from articular cartilage or rib cartilage. Non-limiting examples of articular cartilage may comprise articulating surfaces of the knee, hip, and shoulder joints. In embodiments where the donor is human, bone or osteochondral tissue may be harvested from an articular cartilage region including, but not limited to, the femoral condyle, tibial plateau, femoral head, patella, humeral head, distal tibia, femoral trochlea, talus, or acetabulum. A harvested bone or osteochondral tissue may be the appropriate shape for repairing the relevant bone or osteochondral defect.

(d) Combining a Modified Bone or Osteochondral Tissue with Osteogenic Agents Prior to Implantation

In some embodiments, a modified bone or osteochondral tissue may be combined with one or more osteogenic agents using techniques known in the art. By way of non-limiting example, osteogenic agents include cells, growth factors or matrix proteins that promote bone regeneration after implantation. In certain embodiments, the modified bone or osteochondral tissue may be incubated with various osteogenic agents, directly seed with osteogenic agents, or combined with osteogenic agents using centrifugation, vacuum, or sonication.

In certain embodiments, a modified bone or osteochondral tissue may be combined with one or more osteogenic cells isolated from the intended recipient of the modified bone or osteochondral tissue. In other embodiments, a modified bone or osteochondral tissue may be incubated with one or more osteogenic cells from sources other than the intended recipient of the modified bone or osteochondral tissue. In some embodiments, a modified bone or osteochondral tissue may be incubated with chondrocytes, osteoblasts, bone marrow cells, mesenchymal stem cells, adipose derived stem cells, amniotic stem cells, progenitor cells, or combinations thereof, etc. For instance, a modified bone or osteochondral tissue may be incubated with mesenchymal stem cells from various sources including bone marrow aspirate, adipose tissue, and peripheral blood.

In certain embodiments, a modified bone or osteochondral tissue may be further combined with osteogenic agents meant to improve modified bone or osteochondral tissue function in the recipient. Suitable osteogenic agents are known in the art and may include allogenic cells, osteoconductive factors, and osteoinductive factors. For instance, in some embodiments, a modified bone or osteochondral tissue may be combined with one or more growth factors. Non-limiting growth factors may include, but are not limited to, transforming growth factor-beta (TGFβ), fibroblast growth factor (FGF) (e.g., FGF2, FGF5), bone morphogenetic protein (BMP) (e.g., BMP2, BMP4, BMP6, BMP7), platelet derived growth factor (PDGF), and insulin-related growth factor (IGF) (e.g., IGF1, IGF2).

In certain embodiments, the one or more osteogenic agent may be combined into the modified bone or osteochondral tissue to fill the depleted bone marrow voids in the bone.

In certain embodiments, as discussed herein, a modified bone or osteochondral tissue of the disclosure may include bone channels, including nano-channels, micro-channels, or a combination of nano-channels and micro-channels. In certain aspects of the disclosure, such bone channels may be incubated with osteogenic agents, and these cells may further facilitate in vivo integration of the modified bone or osteochondral tissue upon implantation into a patient.

### (e) Storage of Modified Bone or Osteochondral Grafts

Suitable storage solutions for modified bone or osteochondral tissues are well known to those of ordinary skill in the art, and such solutions may be readily selected and employed by those of skill in the art without undue experimentation. In some embodiments, a modified bone or osteochondral tissue may be frozen at about −20° C. to about −100° C., preferably at about −70° C. In other embodiments, a modified bone or osteochondral tissue may be fresh and may be stored at 4° C.−37° C., preferably 4° C.

A modified bone or osteochondral tissue may be stored, ex vivo, for longer than non-modified controls. One mechanism for assessing storage time is measuring chondrocyte viability and/or cartilage matrix degradation. Methods of measuring chondrocyte viability are known in the art.

Furthermore, methods of the present disclosure encompass methods for improving viability of chondrocytes in a bone or osteochondral graft compared to a non-modified control graft. The methods comprise depleting a bone or osteochondral graft of residual lipids. As noted above, methods of measuring chondrocyte viability are known in the art.

### (f) Repairing a Bone or Osteochondral Defect

Another aspect of the present disclosure encompasses methods for repairing a bone or osteochondral defect in a subject. The method comprises implanting a modified bone at the site of a bone defect in a subject, or implanting an osteochondral tissue at the site of an osteochondral defect in a subject, wherein at least 15%, at least 20%, at least 25%, etc. of the lipids in the bone or osteochondral tissue have been depleted compared to a non-modified bone or osteochondral tissue.

In some embodiments, methods for repairing a bone or osteochondral defect in a subject include implanting a modified bone or osteochondral tissue at the site of an osteochondral defect, wherein the modified bone or osteochondral tissue is combined with one or more osteogenic agents that promote bone regeneration after implantation.

### (i) Types of Defects

In accordance with aspects of this disclosure, any type of bone defect known in the art may be corrected using the modified bone or osteochondral tissue of the disclosure. In certain embodiments, a traumatic defect may be repaired with a modified bone or osteochondral tissue described herein. As used herein, a “traumatic defect” refers to a well delineated defect, usually caused by events compromising trauma, osteochondritis dissecans, or osteonecrosis. In other embodiments, a degenerative defect may be repaired with a modified bone or osteochondral tissue described herein. As used herein, a “degenerative defect” refers to a poorly demarcated defect, and may be caused, for instance, by ligament instability, meniscal injuries, or osteoarthritis. In other embodiments, the methods of the disclosure may be used in connection with the correction of any type of bone or osteochondral defect known in the art, e.g., fusions, osteosarcomas, etc.

Methods of implanting modified bone or osteochondral tissue to repair a bone defect or an osteochondral defect are known in the art. It is appreciated that one skilled in the art would be able to select an appropriate method for implanting a modified bone or osteochondral tissue for purposes of the present disclosure.

In some embodiments, a modified bone or osteochondral tissue is used to repair a bone defect in a subject. In other embodiments, a modified osteochondral tissue is used to repair an osteochondral defect in a subject. In certain embodiments, a modified bone or osteochondral tissue used to repair a defect in a subject may be depleted of residual bone marrow. For instance, a modified bone or osteochondral tissue used to repair a defect in a subject may have at least about 15%, at least about 20%, at least about 25%, at least about 35%, at least about 45%, at least about 55%, at least about 65%, at least about 75%, at least about 85%, or at least about 95% of residual bone marrow in the modified bone or osteochondral tissue removed compared to a control that is not treated by a method described herein.

Suitable subjects may include animals in need of repair of a bone or osteochondral defect. In certain embodiments, suitable subjects include humans. In other embodiments, suitable subjects include companion animals or livestock animals. In particular embodiments, suitable subjects may include equines.

### (ii) Possible Implantation Sites of a Modified Bone or Osteochondral Tissue

A modified bone or osteochondral tissue may be implanted at a site of a defect or an injury in cartilage, a bone, a ligament, a tendon, a meniscus, or a joint. In some embodiments, a method of the present disclosure may encompass implanting a modified bone or osteochondral tissue to repair a joint defect. In certain embodiments, the joint defect may be in a weight-bearing joint. In particular embodiments, a defect suitable for repair with a method of the present disclosure may be located in a shoulder, leg, knee, ankle, hip, foot, pelvis, or spine.

(iii) Operative Treatments

A method of the present disclosure may be used to repair a defect in the same subject that served as a donor for the modified bone or osteochondral tissue (e.g. an autologous graft). Alternatively, a method of the present disclosure may be used to repair a defect in a different subject that the donor of the modified bone or osteochondral tissue, and the donor and the subject are from the same species (e.g. an allograft). In another embodiment, a method of the present disclosure may be used to repair a defect in a different species than the donor of the modified bone or osteochondral tissue (e.g. a xenograft). As described above, suitable xenograft donors may include canine, caprine, swine, or equine donors.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1 Preparation of Osteochondral Allografts by Digestion with Lipase

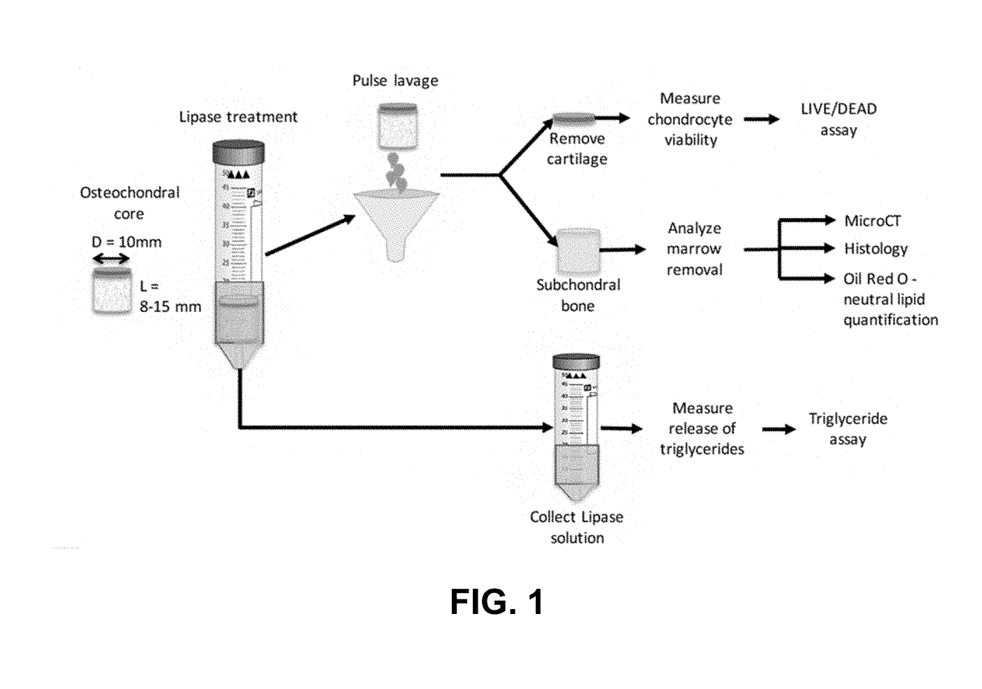

Osteochondral cores (diameter=8-10 mm, bone thickness=8-10 mm) were harvested from the left and right medial trochlear ridge of stifle joints from recently-deceased adult horses within 24 hours post-mortem using Arthrex Osteochondral Allograft Transplantation System (OATS) instrumentation, standard surgical instrumentation used for human patients clinically. The fresh osteochondral cores were incubated in an aqueous solution that contains physiological buffers with the lipase enzymes (10 mg/mL) Porcine pancreatic lipase, Candida rugosa lipase, and Geotrichum candidum lipase. During the 4 hour incubation at 37° C. in the lipase solution, the osteochondral sample was moderately agitated using a shaker plate or nutator mixer to provide homogenous distribution of the lipase throughout the osteochondral graft. Following lipase incubation, the osteochondral cores were soaked and washed by traditional lavage with saline at a flow rate of 1.5 L/min. FIG. 1 is an illustration of the process described in Example 1.

### Example 2 Lipase Enhances Removal of Residual Adipose Marrow from Osteochondral Tissue

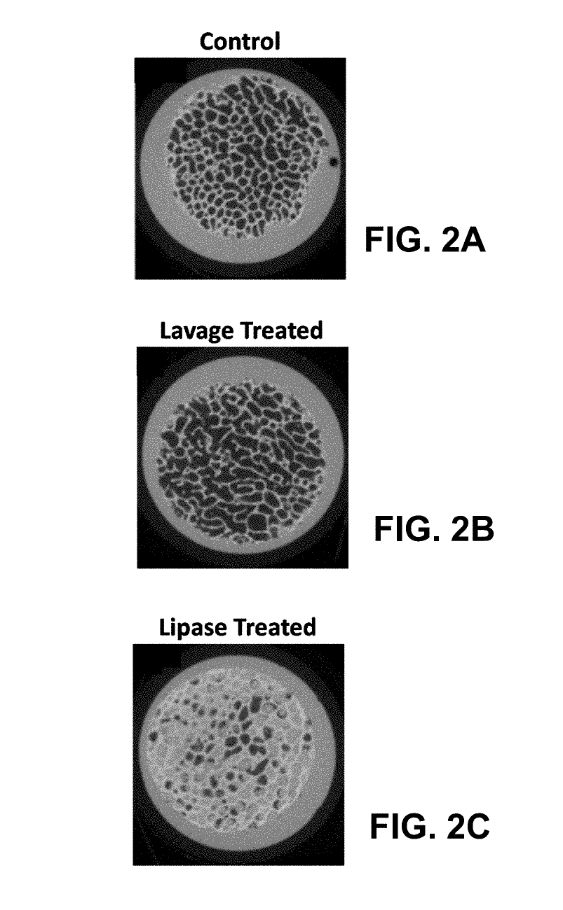

Fresh osteochondral cores were 1) untreated (CTRL); 2) immediately washed by lavage with saline at a flow rate of 1.5 L/min (LAV); or 3) incubated in a lipase solution prior to washing by lavage with saline at a flow rate of 1.5 L/min (LIP). Cartilage was removed from prepared osteochondral cores to measure chondrocyte viability at the surface (en face section) and through the depth (vertical section) by applying LIVE/DEAD reagents for 1 hour at 37° C. in a humidified 5% CO₂ incubator followed by imaging using fluorescence microscopy. In this example, the en face region of the cartilage shows similar chondrocyte viability following treatment by LIP or LAV. Subchondral bone was removed from prepared osteochondral cores to analyze removal of adipose marrow from the cores. The extent of marrow removal was determined by gross visualization, contrast-enhanced microCT, and histology with Oil Red O and hematoxyline staining. Gross cross-sectional images of the bone region showed primarily yellow coloration in the CTRL and LAV treatment groups, indicating high presence of fatty marrow. The LIP group showed increasingly white coloration suggesting greater removal of fatty marrow elements. Histology with Oil Red O staining confirmed that the residual marrow was primarily lipid. MicroCT images of LIP treated samples showed increased contrast infiltration indicating greater adipose marrow removal compared to LAV and CTRL (FIG. 2).

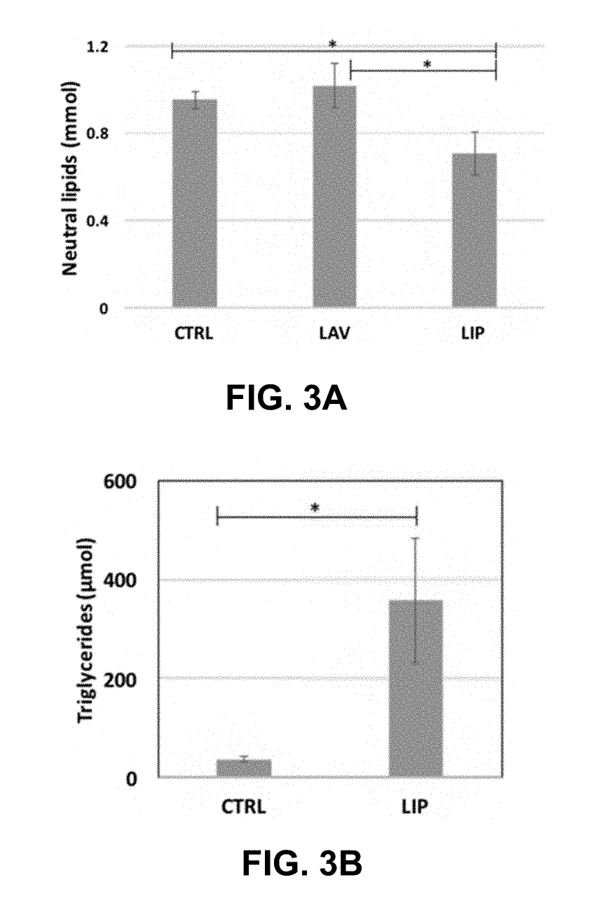

To quantify the amount of residual lipid following treatment, Oil red O extraction and quantification was used. Lipase-treated osteochondral cores show significantly less neutral lipid content compared to the control and lavage-treated groups (FIG. 3A). After osteochondral cores were removed from the lipase treatment, the solution was collected to measure released lipid components using a triglyceride assay. Released lipid components were measured with triglycerides assay. Total residual lipids from the bone were also quantified by extracting lipid with chloroform:methanol and measuring the weight of extracted lipid. (FIG. 3B). LIP-treated osteochondral cores showed significantly more triglyceride release into cleansing solution compared to CTRL cores.

### Example 3 Released Lipid Components in Subchondral Bone May Promote Chondrocyte Death

The availability of osteochondral allografts for clinical use is limited due in part by the decline in chondrocyte viability during prolonged cold storage (4° C.). Osteochondral cores were harvested as decided above and incubated in storage media with increasing warming conditions (4, 24, and 37° C.) to accelerate the release of lipids from the subchondral bone. Warming conditions reduced chondrocyte viability both in the en face views and vertical sections compared to cores tested after 28 days of cold storage. To investigate if the release of lipid components from subchondral bone contributes to the lipotoxic effects that cause chondrocyte death, the release of nitric oxide was measured. Lipid release in osteochondral tissues incubated at 37° C. resulted in higher (p<0.05) nitric oxide production compared to osteochondral tissues from cold storage (4° C.). These results support the idea that removing adipose marrow elements by lipase prior to cold store may extend the viability of chondrocytes by reducing the potential for lipotoxic events.

### Example 4 Fabrication of Bone Nano- and Micro-Sized Channels in Osteochondral Cores

Osteochondral cores (diameter=8-10 mm, bone thickness=8-10 mm) may be harvested from the left and right medial trochlear ridge of stifle joints from recently-deceased adult horses within 24 hours post-mortem using Arthrex Osteochondral Allograft Transplantation System (OATS) instrumentation, standard surgical instrumentation used for human patients clinically. Following harvest, fresh grafts may be stored in clinically relevant storage conditions, which includes standard storage medium containing DMEM and supplements in 4° C. Using laser fabrication, osteochondral cores with channels in the transverse, radial, perpendicular as well as defined orientation through trabeculae of various diameters between 50 nm-100 μm may be prepared (FIG. 4). Micro-CT and morphometric analysis may be performed to measure uniformity of micro-channel diameter, accuracy of placement, and effects on surrounding bone. The μCT may be performed at (9 μm)³ voxel size to provide sufficient resolution.

### Example 5 Intra-Operative Seeding of Autologous Cells on Adipose Marrow-Depleted Osteochondral Allografts

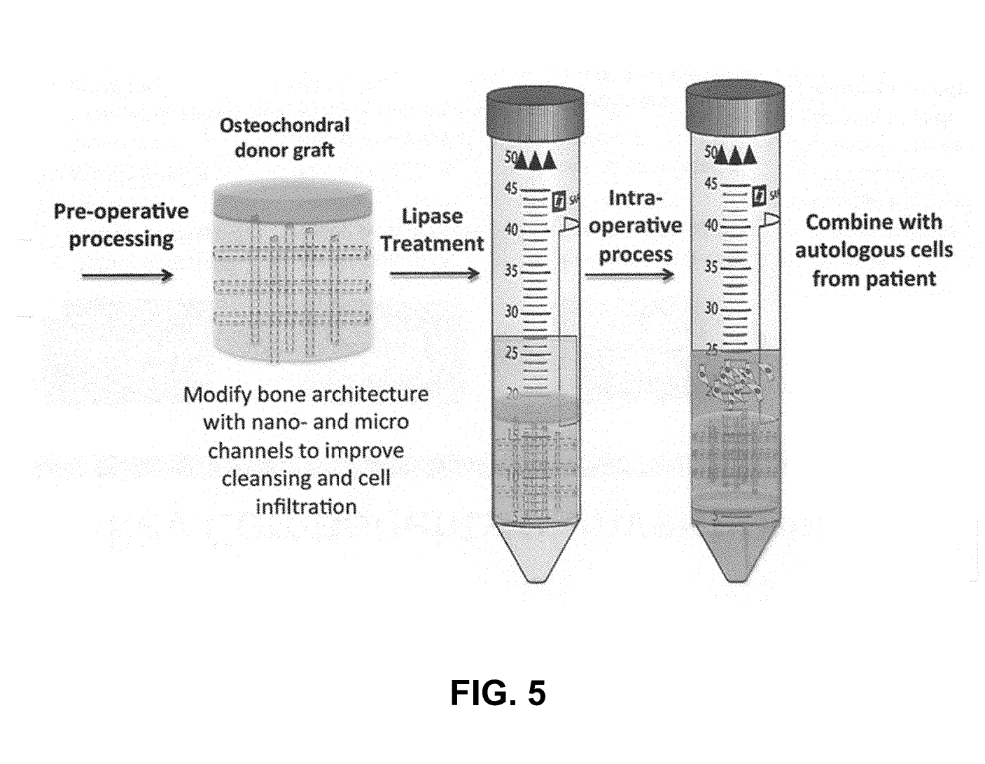

Autologous cells, which include stem cells from various sources including bone marrow aspirate and adipose tissue, were isolated from the patient. Lipase-treated osteochondral cores with and without micro-channels were then combined with the patient's isolated autologous cells to allow for cell infiltration and proliferation. Following significant incubation, the graft will be implanted. FIG. 5 provides an illustration of the methods described in Example 5.

What is claimed is:

1. A modified ex-vivo bone or osteochondral tissue, wherein at least 25% of the residual lipids in the bone or osteochondral tissue have been depleted compared to a non-modified bone or osteochondral tissue.

2. The modified ex-vivo bone or osteochondral tissue of claim 1, wherein the residual lipids are residual adipose marrow.

3. The modified ex-vivo bone or osteochondral tissue of claim 1, wherein the bone or osteochondral tissue is combined with one or more osteogenic agents.

4. The modified ex vivo bone or osteochondral tissue of claim 1, wherein the bone or osteochondral tissue comprises one or more lipase distribution structural features selected from the group consisting of holes, perforations and bone channels.

5. The modified ex vivo bone or osteochondral tissue of claim 4, wherein the one or more lipase distribution structural features are formed in an array of longitudinal and radial structural features.

6. A method of modifying a bone or osteochondral tissue, the method comprising incubating the bone or osteochondral tissue with one or more lipases under conditions suitable for activity of the one or more lipases, wherein after incubation, at least 25% of the residual lipids in the modified bone or osteochondral tissue are depleted compared to a non-modified bone or osteochondral tissue.

7. The method of claim 6, wherein the residual lipids are residual adipose marrow.

8. The method of claim 6, wherein the lipases are selected from the group consisting of mammalian pancreatic lipase, Candida rugosa lipase, Geotrichum candidum lipase, and Chromobacterium viscosum lipase.

9. The method of claim 6, the method further comprising inhibiting the one or more lipases after incubating the bone or osteochondral tissue with the one or more lipases.

10. The method of claim 6, wherein ex vivo storage time of the modified bone or osteochondral tissue is increased in relation to a non-modified bone or osteochondral tissue, wherein storage time is assessed by measuring chondrocyte viability.

11. The method of claim 6, wherein the viability of chondrocytes in the modified bone or osteochondral tissue is increased in relation to a non-modified bone or osteochondral tissue t.

12. The method of claim 6, wherein the method further comprises increasing lipase penetration into the bone or osteochondral tissue by a technique selected from the group consisting of: applying centrifugation to the bone or osteochondral tissue during incubation with the lipase, applying vacuum to the bone or osteochondral tissue during incubation with the lipase, applying sonication to the bone or osteochondral tissue during incubation with the lipase, forming bone lipase distribution structural features in the bone or osteochondral tissue prior to incubating with the lipase, and combination thereof.

13. A method of repairing a bone or osteochondral defect in a subject, the method comprising implanting a modified bone at the site of a bone defect in the subject, or implanting an osteochondral tissue at the site of an osteochondral defect in the subject, wherein at least 25% of the residual lipids in the bone or osteochondral tissue have been depleted compared to a non-modified bone or osteochondral tissue.

14. The method of claim 13, wherein the modified bone or osteochondral tissue comprises one or more lipase distribution structural features selected from the group consisting of holes, perforations and bone channels.

15. The method of claim 14, wherein the one or more lipase distribution structural features are formed in an array of longitudinal and radial structural features.

16. The method of claim 13, wherein the defect is a traumatic or degenerative osteochondral defect.

17. The method of claim 13, wherein the modified bone or osteochondral tissue is combined with one or more osteogenic agents that promote bone regeneration after implantation.

18. The method of claim 13, wherein the modified bone or osteochondral tissue is stored ex vivo prior to implantation.

19. The method of claim 13, wherein the modified bone or osteochondral tissue is autologous to the subject.

20. The method of claim 13, wherein the modified bone or osteochondral tissue is allogeneic to the subject.
